(19) United States
(12) Patent Application Publication
(10) Pub. No.: US 2024/0139551 A1
(43) Pub. Date: May 2, 2024

(54) Transducer Probe for Direct Ultrasonic Neuromodulation and Measurement

(71) Applicant: Alien Sandbox, LLC, Austin, TX (US)

(72) Inventor: Neal A. Hall, Austin, TX (US)

(21) Appl. No.: 18/496,252

(22) Filed: Oct. 27, 2023

Related U.S. Application Data
(60) Provisional application No. 63/420,455, filed on Oct. 28, 2022.

(51) Int. Cl. A61N 7/00

(52) U.S. Cl. CPC A61N 7/00; A61N 2007/0026; A61N 2007/0082

(57) ABSTRACT

Systems, methods, and mechanisms for a transducer probe for ultrasonic measurement and direct neuromodulation, e.g., a transducer probe generating ultrasound across a broad range of frequencies to acoustically modulate groups of neurons. A probe may be configured to generate focused ultrasound inside an acoustic medium. The probe may include a plurality of ultrasonic transducer elements along its length. The probe may be configured to generate ultrasound across a broad range of frequencies. The probe may be configured to focus ultrasound down to an approximately 100 micrometers (μm) diameter spot size, e.g., to a focal point or spot of approximately 100 μm. In addition, the focal spot may be movable in space via phased array focusing.

### PRIORITY DATA

This application claims benefit of priority to U.S. Provisional Application Ser. No. 63/420,455, titled “Transducer Probe for Ultrasonic Measurement and Direct Neuromodulation”, filed Oct. 28, 2022, which is hereby incorporated by reference in its entirety as though fully and completely set forth herein.

### FIELD OF THE INVENTION

This disclosure relates generally to systems, methods, and mechanisms for a transducer probe for ultrasonic measurement and direct neuromodulation, e.g., a transducer probe generating ultrasound across a broad range of frequencies to acoustically modulate groups of neurons.

### DESCRIPTION OF THE RELATED ART

Nearly 100 million Americans are affected by psychiatric and neurological disorders with a significant burden to society costing more than $765 billion dollars. Many of these disorders have limited treatment options and disease remission is not always achieved. Thus, there is a critical need for new innovative methods and technologies for precise neuromodulation of discrete brain circuits involved in these diseases.

In existing implementations, electrical deep brain stimulation (DBS) is an established clinical technique in which a probe, approximately 1 mm in diameter, is embedded into a brain. Electrical signals, generated via the probe, create an electric field in the brain that modulates neural activity. The electrical signals, or electrical impulses, may regulate abnormal impulses in the brain and/or may affect certain cells and chemicals within the brain. Stimulation level is often controlled via a controller (e.g., a pacemaker-like device) implanted under a patient's skin in the upper left chest. A wire, also embedded in the patient, travels from the controller to the probe (or probes). DBS is commonly used to treat numerous conditions such as Parkinson's disease, essential tremor, dystonia, epilepsy, and obsessive-compulsive disorder. In addition, DBS is being studied as a potential treatment for Tourette syndrome, Huntington's disease and chorea, chronic pain, and cluster headache.

The probe may be made up of multiple electrodes that allow for different electric field geometries to be generated in the brain. The various electric field geometries may be controlled to target various regions of the brain for stimulation. Signals generated by the probe may be static voltages (e.g., direct current (DC) voltages) or dynamic voltages (e.g., alternating current (AC) voltages). However, focusing, control, and/or steering of the electric field is limited. Therefore, further improvements in the field are desired.

### SUMMARY OF THE INVENTION

Various embodiments of systems, methods, and mechanisms for a transducer probe for ultrasonic measurement and direct neuromodulation, e.g., a transducer probe generating ultrasound across a broad range of frequencies to acoustically modulate groups of neurons, are described herein.

For example, in some embodiments, a needle-style ultrasonic transducer probe may be configured to generate focused ultrasound inside living brains. The probe may have a diameter of approximately 0.5 millimeters (mm) and include a plurality of ultrasonic transducer elements along its length. Each of the plurality of ultrasonic transducer elements may be individually addressable (e.g., each of the plurality of ultrasonic transducer elements may be independently controlled to produce soundwaves). The probe may be approximately rigid and/or may be flexible. The probe may be configured to generate ultrasound across a broad range of frequencies. The probe may be configured to focus ultrasound down to an approximately 100 micrometers (μm) diameter spot size, e.g., to a focal point or spot of approximately 1100 μm. In addition, the focal spot may be movable in space via phased array focusing. Thus, the probe may be configured to acoustically modulate very small groups of neurons with high spatial resolution in a much more controlled manner and across a broader set of controlled input parameters (e.g., frequency, amplitude, and location) than is possible with head-mounted focused transducers.

As another example, in some embodiments, ultrasound may be radiated through an acoustic medium, e.g., via an array of ultrasonic transducers that may be spaced longitudinally and/or radially about a cylindrical probe, such as a needle-style probe. Further, neural activity may be sensed responsive to the radiated ultrasound, e.g., via local field potential sensors (e.g., one or more local field potential sensors). In addition, an electrical field in the acoustic medium may be generated, e.g., via one or more electrical transducers and the neural activity sensed responsive to the radiated ultrasound may also include neural activity sensed responsive to the generated electrical field, e.g., via local field potential sensors.

As a further example, in some embodiments, a probe may include an array of ultrasonic transducers and local field potential sensors. A probe diameter (and/or diameter of the probe) may be approximately one twenty fourth of a probe length. In addition, the probe may include. The probe may be approximately and/or substantially rigid, e.g., at least in comparison to the acoustic medium and may be made of (e.g., comprised of) acoustically active material. Additionally, each ultrasonic transducer of the array of ultrasonic transducers may be and/or include an electrode pair. Further, the probe may be configured to radiate, via the array of ultrasonic transducers, ultrasound through an acoustic medium and sense, via the local field potential sensors, neural activity responsive to the radiated ultrasound. Note that a location and/or size of a focal spot or region of the radiated ultrasound may be based, at least in part, on input signals to each ultrasonic transducer in the array of ultrasonic transducers. Further, the ultrasonic probe may include a multi-lumen tube and the array of ultrasonic transducers may be comprised on a substrate that resides in an interior of the multi-lumen tube. Additionally, a volume between the substrate and an interior wall of the multi-lumen tube may be filled with an acoustic substance configured to provide acoustic coupling between the substrate and the multi-lumen tube.

As yet another example, in some embodiments, ultrasound may be radiated through an acoustic medium, e.g., via an array of ultrasonic transducers positioned within the acoustic medium and neural activity may be monitored responsive to the radiated ultrasound, e.g., via one or more sensing electrodes positioned on the probe. The neural activity may be associated with patient behavior, at least in some instances. In addition, a location and/or size of a focal spot or region of the radiated ultrasound may be steered, e.g., based on the monitoring and a treatment zone, e.g., a group and/or volume of neurons, within the acoustic medium may be identified, e.g., based on the steering. In other words, as the location and/or size of the focal spot or region of the radiated ultrasound is steered through the acoustic medium, patient behavior/response may be monitored (e.g., via sensed neural activity and/or observation) to determine and/or identify the group and/or volume of neurons for treatment.

This Summary is intended to provide a brief overview of some of the subject matter described in this document. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The following detailed description makes reference to the accompanying drawings, which are now briefly described.

FIGS. 1A and 1B illustrate a possible example of a needle-style ultrasonic transducer probe, according to some embodiments.

FIG. 2 illustrates another possible example of a probe, according to some embodiments.

FIGS. 3A, 3B, and 3C illustrate further possible examples of a probe, according to some embodiments.

FIGS. 4A and 4B illustrate a unidirectional end-fire pattern at 0.5 MHz, pointing along the axis of the probe, according to some embodiments.

FIG. 5A illustrates a broadside pattern for a planar MEMS array integrated into one side of a probe, according to some embodiments.

FIG. 5B illustrates cross sections of the field intensity near the focal point, according to some embodiments.

FIG. 6 illustrates a broadside beam steering simulation, according to some embodiments.

FIG. 7 illustrates an example of a cross-section of a probe in which an acoustically active material resides on an exterior of the probe, according to some embodiments.

FIG. 8 illustrates an example of a cross-section of a probe that includes acoustically active tubing, according to some embodiments.

FIG. 9 illustrates an example of a cross-section of a probe in which acoustically active material and electrodes reside inside a lumen of a multi-lumen tubing, according to some embodiments.

FIG. 10 illustrates an example of a cross-section of a probe in which acoustically active material resides in a central lumen.

FIGS. 11 and 12 illustrate block diagrams of examples of methods for intra-acoustic medium ultrasonic neuromodulation, according to some embodiments.

While the invention is susceptible to various modifications and alternative forms, specific embodiments thereof are shown by way of example in the drawings and are herein described in detail. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE INVENTION

### Terms

The following is a glossary of terms used in this disclosure:

Memory Medium—Any of various types of non-transitory memory devices or storage devices. The term “memory medium” is intended to include an installation medium, e.g., a CD-ROM, floppy disks, or tape device; a computer system memory or random-access memory such as DRAM, DDR RAM, SRAM, EDO RAM, Rambus RAM, etc.; a non-volatile memory such as a Flash, magnetic media, e.g., a hard drive, or optical storage; registers, or other similar types of memory elements, etc. The memory medium may include other types of non-transitory memory as well or combinations thereof. In addition, the memory medium may be located in a first computer system in which the programs are executed, or may be located in a second different computer system which connects to the first computer system over a network, such as the Internet. In the latter instance, the second computer system may provide program instructions to the first computer for execution. The term “memory medium” may include two or more memory mediums which may reside in different locations, e.g., in different computer systems that are connected over a network. The memory medium may store program instructions (e.g., embodied as computer programs) that may be executed by one or more processors.

Carrier Medium—a memory medium as described above, as well as a physical transmission medium, such as a bus, network, and/or other physical transmission medium that conveys signals such as electrical, electromagnetic, or digital signals.

Functional Unit (or Processing Element/Processor)—refers to various elements or combinations of elements. Processing elements include, for example, circuits such as an ASIC (Application Specific Integrated Circuit), portions or circuits of individual processor cores, entire processor cores, individual processors, programmable hardware devices such as a field programmable gate array (FPGA), and/or larger portions of systems that include multiple processors, as well as any combinations thereof.

Processing Element/Processor (or Functional Unit)—refers to various elements or combinations of elements. Processing elements include, for example, circuits such as an ASIC (Application Specific Integrated Circuit), portions or circuits of individual processor cores, entire processor cores, individual processors, programmable hardware devices such as a field programmable gate array (FPGA), and/or larger portions of systems that include multiple processors.

Programmable Hardware Element—includes various hardware devices comprising multiple programmable function blocks connected via a programmable interconnect. Examples include FPGAs (Field Programmable Gate Arrays), PLDs (Programmable Logic Devices), FPOAs (Field Programmable Object Arrays), and CPLDs (Complex PLDs). The programmable function blocks may range from fine grained (combinatorial logic or look up tables) to coarse grained (arithmetic logic units or processor cores). A programmable hardware element may also be referred to as “reconfigurable logic”.

Computer System (or Computer)—any of various types of computing or processing systems, including a personal computer system (PC), mainframe computer system, workstation, network appliance, Internet appliance, personal digital assistant (PDA), television system, grid computing system, or other device or combinations of devices. In general, the term “computer system” can be broadly defined to encompass any device (or combination of devices) having at least one processor that executes instructions from a memory medium.

Sound/Soundwave—refers to mechanical and/or elastic waves (including surface acoustic waves) in any medium (gas, liquid, solid, gel, soil, and so forth) and/or combination of media, including interfaces between media. Sound may occur at any amplitude and at any frequency, e.g., ranging from infrasound (e.g., sound waves with frequencies below human audibility, generally considered to be under 20 hertz (Hz)) to ultrasound (e.g., sound waves with frequencies above human audibility, generally considered to be above 20 kilohertz (kHz)), as well as all frequencies in between.

Sound Pressure (or Acoustic Pressure)—refers to a local pressure and/or stress deviation from an ambient (quiescent, average, and/or equilibrium) pressure, caused by a sound wave.

Sound Beam—refers to an area through which sound energy emitted from a sound transducer travels. A sound beam may be three dimensional and symmetrical around its central axis. In some instances, e.g., depending on a sound transducer implemented to generate the sound beam, a sound beam may include a converging region and a diverging region. The regions may intersect at a focal point of the sound beam. In other instances, e.g., depending on a sound transducer implemented to generate the sound beam, a sound beam may include a near field (or Fresnel zone) which is cylindrical in shape and a far field (or Fraunhofer zone) which is conical in shape and in which the sound beam diverges. A sound beam may be a continuous wave, single-frequency beam. Additionally, a sound beam may be tone-bursts, e.g., comprised of a finite number of cycles at a particular frequency. Further, a sound beam may be broadband, comprised of a finite duration pulse and having a broad spectral content.

Medium (or Acoustic Medium)—refers to any substance through which sound may pass, including, but not limited to a gas, a liquid, a solid, a gel, soil, and so forth. Example of mediums may include, but are not limited to, various portions of human anatomy, including soft tissue such as muscle (e.g., heart) and organs (e.g., brain, kidney, lungs, and so forth) as well as hard tissue (e.g., such as bones), as well as various layers of the earth, including the crust, the upper mantle, the mantle, the outer core, and the inner core.

Automatically—refers to an action or operation performed by a computer system (e.g., software executed by the computer system) or device (e.g., circuitry, programmable hardware elements, ASICs, etc.), without user input directly specifying or performing the action or operation. Thus, the term “automatically” is in contrast to an operation being manually performed or specified by the user, where the user provides input to directly perform the operation. An automatic procedure may be initiated by input provided by the user, but the subsequent actions that are performed “automatically” are not specified by the user, i.e., are not performed “manually”, where the user specifies each action to perform. For example, a user filling out an electronic form by selecting each field and providing input specifying information (e.g., by typing information, selecting check boxes, radio selections, etc.) is filling out the form manually, even though the computer system must update the form in response to the user actions. The form may be automatically filled out by the computer system where the computer system (e.g., software executing on the computer system) analyzes the fields of the form and fills in the form without any user input specifying the answers to the fields. As indicated above, the user may invoke the automatic filling of the form, but is not involved in the actual filling of the form (e.g., the user is not manually specifying answers to fields but rather they are being automatically completed). The present specification provides various examples of operations being automatically performed in response to actions the user has taken.

Approximately/Substantially—refers to a value that is almost correct or exact. For example, approximately may refer to a value that is within 1 to 10 percent of the exact (or desired) value. It should be noted, however, that the actual threshold value (or tolerance) may be application dependent. For example, in one embodiment, “approximately” may mean within 0.1% of some specified or desired value, while in various other embodiments, the threshold may be, for example, 2%, 3%, 5%, and so forth, as desired or as required by the particular application. Furthermore, the term approximately may be used interchangeable with the term substantially. In other words, the terms approximately and substantially are used synonymously to refer to a value, or shape, that is almost correct or exact.

Couple—refers to the combining of two or more elements or parts. The term “couple” is intended to denote the linking of part A to part B, however, the term “couple” does not exclude the use of intervening parts between part A and part B to achieve the coupling of part A to part B. For example, the phrase “part A may be coupled to part B” means that part A and part B may be linked indirectly, e.g., via part C. Thus, part A may be connected to part C and part C may be connected to part B to achieve the coupling of part A to part B.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must). The words “include,” “including,” and “includes” indicate open-ended relationships and therefore mean including, but not limited to. Similarly, the words “have,” “having,” and “has” also indicated open-ended relationships, and thus mean having, but not limited to. The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “third component electrically connected to the module substrate” does not preclude scenarios in which a “fourth component electrically connected to the module substrate” is connected prior to the third component, unless otherwise specified. Similarly, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a set of electrical conductors may be configured to electrically connect a module to another module, even when the two modules are not connected). In some contexts, “configured to” may be a broad recitation of structure generally meaning “having circuitry that” performs the task or tasks during operation. As such, the component can be configured to perform the task even when the component is not currently on. In general, the circuitry that forms the structure corresponding to “configured to” may include hardware circuits.

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112(f) interpretation for that component.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

### Transducer Probe for Ultrasonic Measurement and Neuromodulation

In current implementations, electrical deep brain stimulation (DBS) is an established clinical technique in which a probe, approximately 1 mm in diameter, is embedded into a brain. Electrical signals, generated via the probe, create an electric field in the brain that modulates neural activity. The electrical signals, or electrical impulses, may regulate abnormal impulses in the brain and/or may affect certain cells and chemicals within the brain. Stimulation level is often controlled via a controller (e.g., a pacemaker-like device) implanted under a patient's skin in the upper left chest. DBS is commonly used to treat numerous conditions such as Parkinson's disease, essential tremor, dystonia, epilepsy, and obsessive-compulsive disorder. In addition, DBS is being studied as a potential treatment for Tourette syndrome, Huntington's disease and chorea, chronic pain, and cluster headache. The probe may be made up of multiple electrodes that allow for different electric field geometries to be generated in the brain. The various electric field geometries may be controlled to target various regions of the brain for stimulation. Signals generated by the probe may be static voltages (e.g., direct current (DC) voltages) or dynamic voltages (e.g., alternating current (AC) voltages). However, control, focusing, and/or steering of the electric field is limited.

Low-intensity focused ultrasound (LIFU) is another novel neuromodulatory approach that uses mechanical energy to modulate neuronal activity with high spatial resolution and adjustable depth of focus. LIFU has been used safely and effectively for neuromodulation in various ranges of animals, including mice, rats, rabbits, sheep, pigs, and primates. LIFU has also been an effective method for transient cortical and sub-cortical neuromodulation in humans. However, despite its enormous promise, there are considerable gaps that preclude LIFU from immediate translation to human clinical treatment options. Critically, the effect of LIFU on individual neurons in humans is unknown. LIFU modulation may have varying effects depending on neuronal type and activity, brain region, and also the particular waveform parameters applied. Thus, there is a need to understand how ultrasound interacts with neurons in the human brain so that the technology can be deployed rationally and with predictable clinical effects.

There has been some success with single unit activity in response to LIFU in awake humans during movement disorder surgeries where implanted electrodes are able to simultaneously record the responses from single neurons and their local field potentials (LFPs). That study found that 5 minutes of pulsed LIFU changed sub-thalamic nucleus (STN) LFP activity from a baseline of approximately 20 Hz to approximately 30 Hz that persisted upon cessation of LIFU. This finding is similar to research exploring how dopaminergic agents affect STN firing in Parkinson's disease. Despite these significant results, currently LIFU is delivered through the skull using a 500 kHz single element that precludes sub-millimeter resolution and precise control of beam magnitude and geometry—as the skull is the primary barrier to ultrasound. Therefore, improvements are desired.

Embodiments described herein provide systems, methods, and mechanisms for a transducer probe for ultrasonic measurement and direct neuromodulation, e.g., a transducer probe generating ultrasound across a broad range of frequencies to acoustically modulate groups of neurons. Further, embodiments described herein address various issues related to ultrasonic neuromodulation, including, but not limited to, developing the relationship between neuron behavior and ultrasound, e.g., via enabling a mapping of ultrasonic frequency, ultrasonic intensity, neuron type, and brain region to behavior of individual neurons or small groups of neurons. For example, the ability to stimulate and record in vivo neuronal activity in response to LIFU may provide a fundamental understanding of LIFU neuromodulation from the single neuron to brain circuit and ultimately to behavior. Such information may lay a foundation to use ultrasound diagnostically for brain mapping and therapeutically for targeted psychiatric and neurological interventions and treatments. Benefits of in vivo measurements may include, but are not limited to, direct, real-time recording of neural spiking activity, high sensitivity to measure single (and groups of) neurons activity and response, exquisite spatial and temporal resolution, high signal to noise ratio, no confound of anesthesia (human studies), no confound of skull, and no scalability or translational issues.

Note that the embodiments described herein may aid in advancement of a fundamental scientific understanding of acoustic neuromodulation, e.g., may aid experiments aimed at understanding how and/or why ultrasound modulates neurological activity and understanding the mapping of input parameters (vibration amplitude, frequency, target region and size) to various neurological output parameters. In addition, embodiments described herein may replace and/or advance electrical DBS in humans at least because ultrasonic DBS could overcome issues related to focusing, controlling, and steering of an electric field that occur with electrical DBS.

In some embodiments, sound may be focused using phased array imaging. A focal spot or a focal region may be moved about by controlling input signals using phased array techniques as known to those skilled in the art. In some instances, the focal region may be positioned in close proximity to the probe (e.g., 1 mm from probe axis) or a distance away from the probe (e.g., 2 or 3 cm away). In some instances, the focal region may be steered, e.g., an ultrasound beam emitted from the array can be steered left to right. Additionally, in some instances, multiple focal regions can be generated simultaneously, e.g., selection of driving signals to each ultrasonic transducer can be such that two or three distinct focal regions are generated at the same time and, at least in some instances, in different directions. These multiple focal regions may be moved about in time to achieve a so-called spatiotemporal neuromodulation.

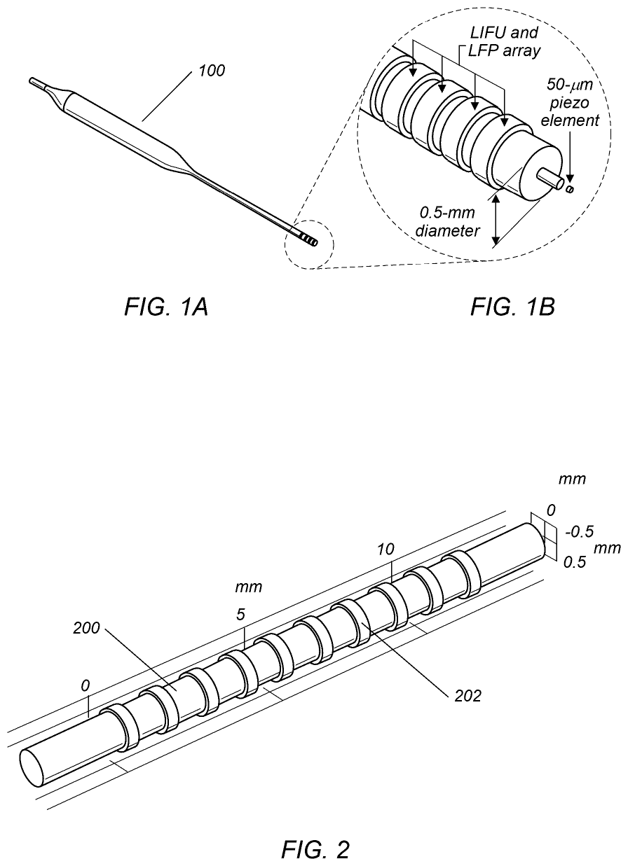

In some embodiments, a needle-style ultrasonic transducer probe may be configured to generate focused ultrasound inside living brains. When inserted in brains of living animals and humans, the probe may generate focused ultrasound in any direction and across a frequency range 0.25 megahertz (MHz) to 5 MHz. In addition, the probe may be configured such that the ultrasound may be focused to a 100 μm diameter spot. In some embodiments, the ultrasound may be focused in the immediate vicinity of the probe, e.g., within 10 percent of a longitudinal length, L, of the probe (e.g., 0.1 L) or as far out as 2 times the longitudinal length of the probe (e.g., 2 L). In some instances, the phased array may be integrated with electrodes configured to measure local field potentials (LFPs). LFPs recordings may be subsequently filtered in frequency to understand which LFP signal components result from synaptic firings of relatively large groups of neurons versus which LFP signal components are due to action potentials of neurons in immediate vicinity of the probe. FIGS. 1A and 1B illustrate a possible example of a needle-style ultrasonic transducer probe, according to some embodiments. Note that needle-style is not meant to imply rigidity. Needle-style is meant to imply a slender aspect ratio. Thus, a needle-style probe may be approximately (or substantially) rigid, nominally rigid, nominally flexible, and/or flexible. As shown, the needle-style ultrasonic transducer probe 100 may include both a low-intensity focused ultrasound (LIFU) array and an LFP array. In addition, in at least some instances, the needle-style ultrasonic transducer probe 100 may further include an ultra-small piezo electric element at its tip that may be configured to stimulate a single cell. As shown, the piezo electric element may be approximately 50 μm, e.g., approximately one-tenth the diameter of the needle-style ultrasonic transducer probe 100.

In some embodiments, the needle-style ultrasonic transducer probe 100 (e.g., probe) may be configured to direct ultrasonic dosing of a single or small group of neurons in living brains and simultaneously perform electrical measurement of the response of those neurons. The probe may be configured to enter a living brain (human or animal) much like an electrical deep brain stimulation (DBS) probe with an exact location of the probe in relation to internal regions of the brain precisely known using X-ray or magnetic resonance imaging (MRI) taken prior to ultrasonic experimentation. Once in the target position, the probe may be configured to generate a focus ultrasound with a high spatially resolved focal point (e.g., approximately 100 μm focal diameter) to excite a small group of neurons. In some instances, for an even higher spatial resolution, an individual transducer element with a 50 μm diameter may be integrated onto the tip of the probe as shown in FIG. 1B. Such a transducer, with a diameter on the order of a single neuron, may vibrate or transmit in either direct contact with or in immediate proximity of a targeted region. As such, direct and highly controllable dosing of a single neuron or a small number of neurons may be possible over a broad and controllable range of frequencies (e.g., such as DC to 10 MHz) and vibration amplitudes.

In addition, the probe illustrated in FIGS. 1A and 1B may also include micro electrodes for measurement of local field potential (LFP) waveforms. Note that current understanding of LFP signals is that LFPs may result from a combination of action potentials from neurons in direct contact with the electrode and from synchronized synaptic currents in larger groups of neurons not in immediate contact with the electrode. The former may be distinguishable from the later using frequency-selective filtering. Additionally, action potentials are known to generate higher frequency spiking as compared to lower frequency waves (below 100 Hz) generated by synaptic currents.

Thus, in at least some embodiments, the probe may combine multiple capabilities, e.g., (1) a focused ultrasound spot with 100 μm diameter may be steered to any point in proximity of the probe (e.g., within 10 L of the probe and (2) electrodes on the probe may measure LFPs resulting from the neuromodulation. Note that an addressable acoustic field may include points in direct contact with the LFP electrodes, e.g., ultrasound may be focused onto a single neuron and LFPs which comprise action potentials from that same neuron may be directly measured.

In some embodiments, the probe may be combined with traditional electrical DBS. For example, the probe may include an ultrasonic transducer array and electrical DBS electrodes that are used to generate an electrical field. Further, in some instances, the probe may also include neuro sensors, such as local field potential sensors. Such a probe may thus include multiple types of neuromodulation (ultrasound and electrical) with integrated neuro sensors. In addition, electrodes along the probe may share various functions. For example, an electrode configured for ultrasonic actuation at an ultrasonic frequency may also be configured to apply an electric field at the same or a different frequency or at DC. In some cases, the two types of neuromodulation may be applied simultaneously, using the same shared electrode. In other words, the shared electrode may be configured for ultrasonic actuation and electric field actuation.

As noted above, in some embodiments, the probe may include a plurality of ultrasonic transducer element, each ultrasonic transducer element individually controllable. Thus, in some embodiments, a focal point of the ultrasound may be configurable (e.g., in both terms of size of the focal point and location of the focal point) to acoustically modulate a small group of neurons, e.g., a group of neurons within an area/volume defined by a 100-micrometer diameter spherical region.

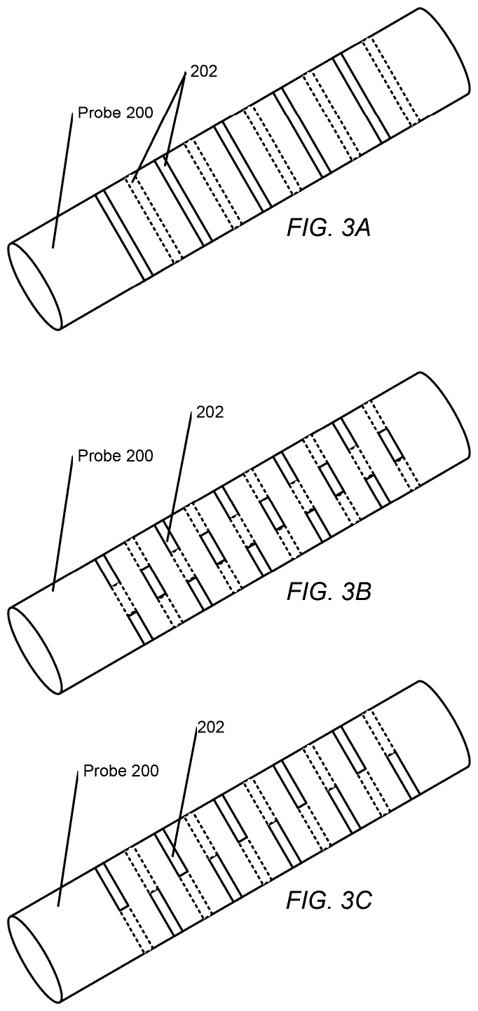

As illustrated by FIG. 2, a probe 200, which may be an example of the needle-style ultrasonic transducer probe 100, for example, may have a diameter of approximately 0.5 millimeters (mm) and include a plurality of ultrasonic transducer elements 202 along its length. For example, the probe 200 may include 10 or more ultrasonic transducer elements 202 along its length. As shown in FIG. 2, the plurality of ultrasonic transducer elements 202 may be arranged along the length of the probe 200. Additionally, as shown in FIGS. 3A, 3B, and 3C, the plurality of ultrasonic transducer elements 202 may also be arranged along the circumference of the probe. For example, as shown in FIG. 3A, the ultrasonic transducer elements 202 may be “split” such that each transducer encompasses half the circumference of the probe 200. Thus, as shown in FIG. 3A, the ultrasonic transducer elements 202 may alternate sides of the probe 200. Note that other geometries are possible as well, e.g., as illustrated by FIG. 3B (ultrasonic transducer elements 202 are “split” such that each transducer element 202 encompasses one-third the circumference of the probe 200) and FIG. 3C (ultrasonic transducer elements are “split” such that each transducer element 202 encompasses one-fourth the circumference of the probe 200). Note that independent of the geometric configuration, each of the plurality of ultrasonic transducer elements 202 may be individually addressable (e.g., each of the plurality of ultrasonic transducer elements may be independently controlled to produce soundwaves) and the probe 200 may be configured to generate ultrasound across a broad range of frequencies.

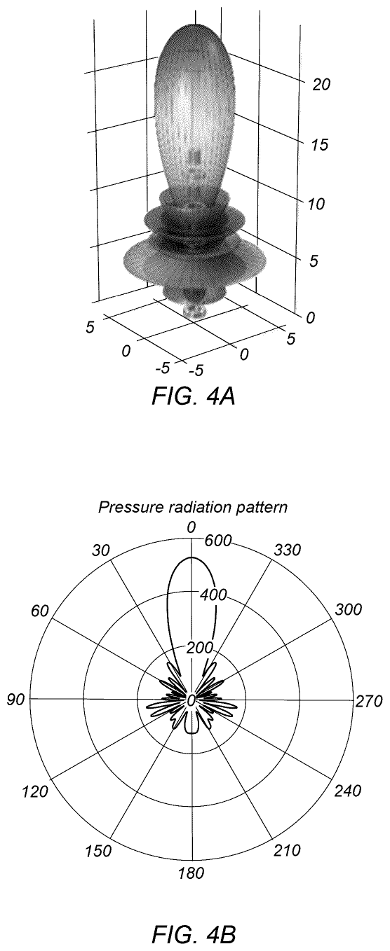

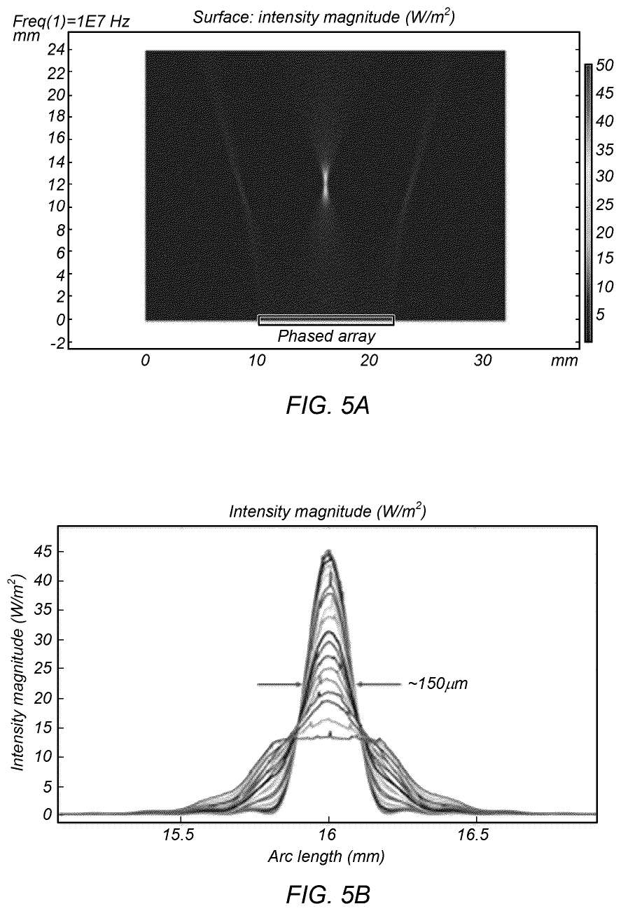

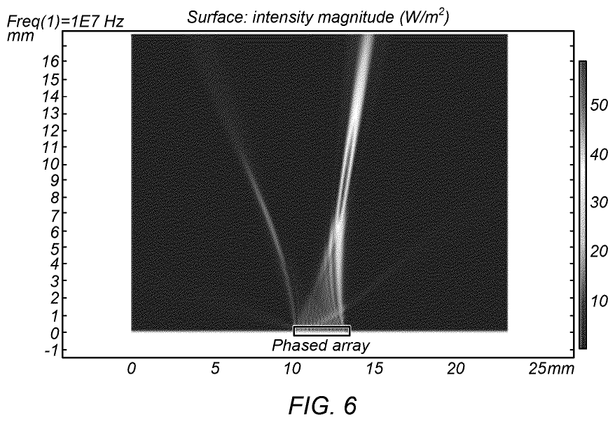

FIGS. 4A and 4B illustrate a unidirectional end-fire pattern at 0.5 MHz, pointing along the axis of a probe, such as probe 200, according to some embodiments. FIG. 5A illustrates a broadside pattern (e.g., a pattern normal to the probe axis) at 10 MHz for a planar MEMS array integrated into one side of a probe, such as probe 200, according to some embodiments. The horizontal axis coincides with the probe 200, and the location of the phased line-array with 12-mm span is labeled. The radiated field is focused to a small spot located approximately 12 mm normal to the probe axis. FIG. 5B illustrates cross sections of the field intensity near the focal point, revealing the 150 μm spot size, according to some embodiments. These figures illustrate a focal spot that is approximately 20 times smaller than is achievable with head-mounted transducers, implying an improvement of 20 times in spatial resolution for neuromodulation. FIG. 6 illustrates a broadside beam steering simulation at 10 MHz, according to some embodiments. Taken together, FIGS. 5B and 6 illustrate an ability to move a highly-resolved focal spot in space without physically moving the probe.

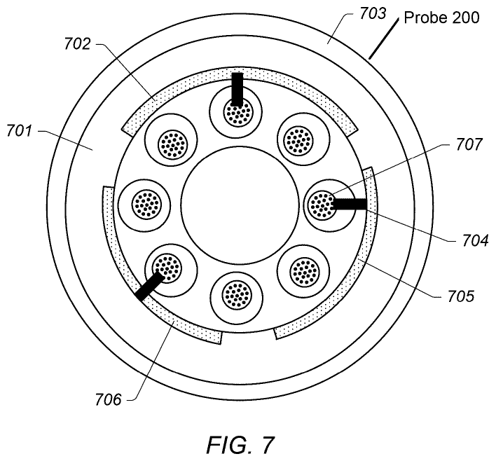

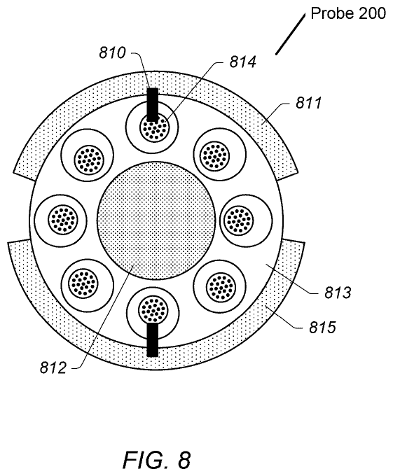

FIG. 7 illustrates an example of a cross-section of a probe, such as probe 200, in which an acoustically active material resides on an exterior of the probe, according to some embodiments. As shown, acoustically active material 701 resides on an exterior of multi-lumen tubing (e.g., the probe). Note that acoustically active material refers to any material that mechanically expands or strains in response to an electrical or magnetic field. These materials may include ferroelectric, piezoelectric, magnetostrictive, and/or piezomagnetic materials. The acoustically active material 701 may be excited and may vibrate in regions between two electrodes of different electrical potential. As shown in FIG. 7, a potential may be applied between outer electrode 703 and inner electrode 702 to actuate a region of the acoustically active material 701 in between these electrodes. The same potential or a different potential may be applied between outer electrode 703 and inner electrode 705 to actuate the acoustically active material 701 filling the space between these electrodes. In this manner, different regions of the acoustically active material 701 may be excited differently. Interconnect 704 may connect electrode 705 to an interior wire 707 and interior wire 707 may route to a controlling unit. Electrodes 702, 705, and 706 may have a finite span along a longitudinal axis of the probe so that separate individually-addressable electrodes may exist at different cross sections of the probe.

FIG. 8 illustrates an example of a cross-section of a probe, such as probe 100, that includes acoustically active tubing, according to some embodiments. As shown, tube material 813 may be acoustically active. A central interior lumen may be filled with wire 812 that serves as an interior electrode and routes down a length of the tube to a control unit. A potential may be applied between exterior electrode 811 and interior electrode 812 to vibrate material 813 in a space between electrodes 811 and 812. Interconnect 810 may connect electrode 811 to wire 814 and wire 814 may route to a controlling unit. Electrodes 811 and 815 may have a finite span along a longitudinal axis of the probe such that separate individually-addressable electrodes may exist at different cross sections of the probe.

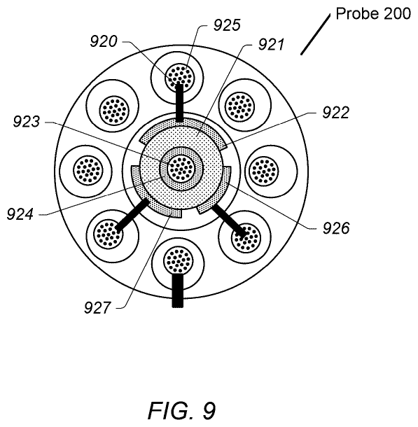

FIG. 9 illustrates an example of a cross-section of a probe, such as probe 200, in which acoustically active material and electrodes reside inside a lumen of a multi-lumen tubing, according to some embodiments. As shown, acoustically active material 921, electrode 924, and electrode 922 may all reside inside the lumen of the multi-lumen tubing. Wire 923 may connect to electrode 924 and may route down a longitudinal length of the probe to a controlling unit. Applying a potential between electrodes 922 and 924 may excite and vibrate acoustically active material 921 in a region between these electrodes. Interconnect 920 connects electrode 922 to wire 925 and wire 925 may route to the controlling unit. Electrodes 922, 926, and 927 may have a finite span along the longitudinal axis of the probe such that separate individually-addressable electrodes may exist at different cross sections of the probe.

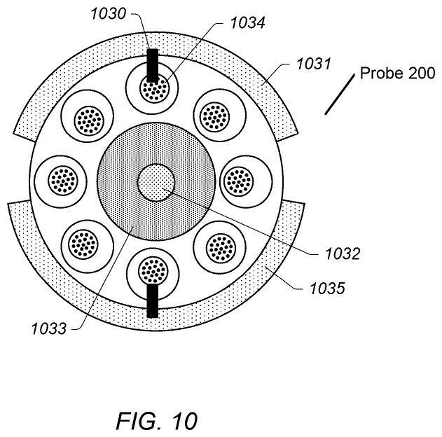

FIG. 10 illustrates an example of a cross-section of a probe, such as probe 200, in which acoustically active material resides in a central lumen. As shown acoustically active material 1033 may reside in the central lumen and wire 1032 may serve an interior electrode and route to a controlling unit. A potential may be applied between electrodes 1031 and 1032 to vibrate acoustically active material 1033 in a region between these two electrodes. Electrode 1031 may connect to wire 1034 and wire 1034 may route to the controlling unit. Electrodes 1031 and 1035 may have a finite span along a longitudinal axis of the probe such that separate individually-addressable electrodes may exist at different cross sections of the probe.

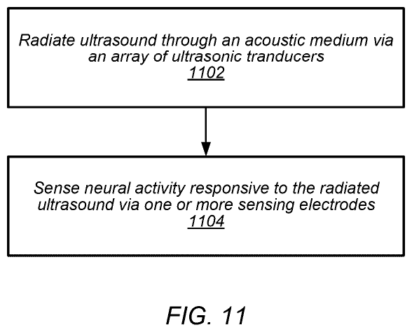

FIG. 11 illustrates a block diagram of an example of a method for intra-acoustic medium ultrasonic neuromodulation, according to some embodiments. The method shown in FIG. 11 may be used in conjunction with any of the systems, methods, or devices shown in the Figures, among other devices. In various embodiments, some of the method elements shown may be performed concurrently, in a different order than shown, or may be omitted. Additional method elements may also be performed as desired. As shown, this method may operate as follows.

At 1102, ultrasound may be radiated through an acoustic medium, e.g., via an array of ultrasonic transducers positioned within the acoustic medium. In some instances, the array of ultrasonic transducers may include (e.g., comprise) at least 10 (e.g., 10 or more) ultrasonic transducers. The ultrasonic transducers in the array of ultrasonic transducers may be spaced longitudinally and/or radially about a cylindrical probe, e.g., a needle-style probe. Additionally, in some instances, each ultrasonic transducer in the array of ultrasonic transducers may be individually addressable. Further, in some instances, each ultrasonic transducer in the array of ultrasonic transducers may be independently controlled to produce the radiated ultrasound.

In some instances, a location and/or a size of a focal spot, point, and/or region of the radiated ultrasound may be based, at least in part, on input signals to each ultrasonic transducer in the array of ultrasonic transducers. In some instances, a location of the focal spot, point, and/or region may be dependent on a length, L, of the array of ultrasonic transducers. For example, the location of the focal spot, point, and/or region may be between 0.1 L and 2 L from the array of ultrasonic transducers. In some instances, a diameter of the focal spot, point, and/or region may be less than 150 micrometers. In some instances, a diameter of the focal spot, point, and/or region may be less than 100 micrometers. In some instances, a frequency of the ultrasound may be between 0.25 MHz to 10 MHz. In some instances, the focal spot, point, and/or region may include (e.g., comprise) a group of neurons.

At 1104, neural activity may be sensed responsive to the radiated ultrasound, e.g., via one or more sensing electrodes positioned within the acoustic medium. The one or more sensing electrodes may be configured to sense local field potentials. Thus, in at least some instances, the one or more sensing electrodes may be one or more local field potential sensors.

In some instances, an electrical field in the acoustic medium may be generated, e.g., via one or more electrical transducers. In such instances, the neural activity sensed responsive to the radiated ultrasound may also include neural activity sensed responsive to the generated electrical field, e.g., via local field potential sensors. In some instances, the one or more electrical transducers may be configured for electrical deep brain stimulation. Further, in some instances, the ultrasonic transducers and the one or more electrical transducers may share at least one electrode.

In some instances, the array of ultrasonic transducers and local field potential sensors may be included (e.g., comprised) on a probe, e.g., on an ultrasonic probe. In some instances, a probe diameter (and/or diameter of the probe) may be approximately one twenty fourth of a probe length. Further, in some instances, the local field potential sensors may also be included (e.g., comprised) on the probe. In some instances, the ultrasonic probe may be flexible. In other instances, the ultrasonic probe may be approximately and/or substantially rigid, e.g., at least in comparison to the acoustic medium. In some instances, the ultrasonic probe may be made, at least partially, of (e.g., comprised, at least partially, of) acoustically active material. In such instances, the ultrasonic probe may further include a multi-lumen tube and the array of ultrasonic transducers may be comprised on a substrate that resides in an interior of the multi-lumen tube. Additionally, a volume between the substrate and an interior wall of the multi-lumen tube may be filled with an acoustic substance configured to provide acoustic coupling between the substrate and the multi-lumen tube. In some instances, an ultrasonic transducer of the array of ultrasonic transducers may include an electrode pair. In other words, each ultrasonic transducer of the array of ultrasonic transducers may be and/or include an electrode pair. In some instances, the ultrasonic probe may include one or more electrical transducers.

In some instances, a location of the focal spot or region of the radiated ultrasound may be on or in immediate proximity of a surface of the probe thereby enabling substantially direct stimulation and excitation of a small group of neurons. In addition, the location may coincide with a location of at least one sensing electrode of the one or more sensing electrodes.

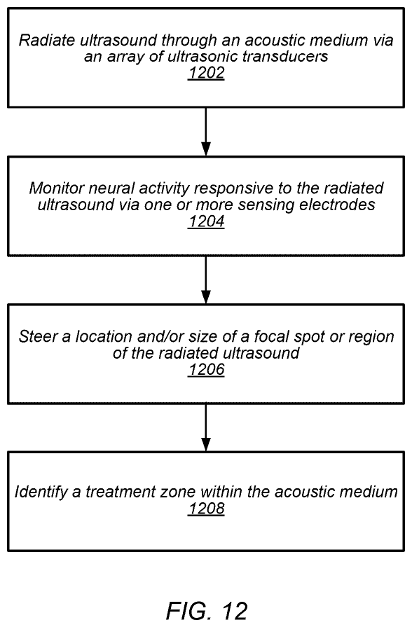

FIG. 12 illustrates a block diagram of another example of a method for intra-acoustic medium ultrasonic neuromodulation, according to some embodiments. The method shown in FIG. 12 may be used in conjunction with any of the systems, methods, or devices shown in the Figures, among other devices. In various embodiments, some of the method elements shown may be performed concurrently, in a different order than shown, or may be omitted. Additional method elements may also be performed as desired. As shown, this method may operate as follows.

At 1202, ultrasound may be radiated through an acoustic medium, e.g., via an array of ultrasonic transducers positioned within the acoustic medium. In some instances, the array of ultrasonic transducers may include (e.g., comprise) at least 10 (e.g., 10 or more) ultrasonic transducers. The ultrasonic transducers in the array of ultrasonic transducers may be spaced longitudinally and/or radially about a cylindrical probe, e.g., a needle-style probe. Additionally, in some instances, each ultrasonic transducer in the array of ultrasonic transducers may be individually addressable. Further, in some instances, each ultrasonic transducer in the array of ultrasonic transducers may be independently controlled to produce the radiated ultrasound.

In some instances, a location and/or a size of a focal spot, point, and/or region of the radiated ultrasound may be based, at least in part, on input signals to each ultrasonic transducer in the array of ultrasonic transducers. In some instances, a location of the focal spot, point, and/or region may be dependent on a length, L, of the array of ultrasonic transducers. For example, the location of the focal spot, point, and/or region may be between 0.1 L and 2 L from the array of ultrasonic transducers. In some instances, a diameter of the focal spot, point, and/or region may be less than 150 micrometers. In some instances, a diameter of the focal spot, point, and/or region may be less than 100 micrometers. In some instances, a frequency of the ultrasound may be between 0.25 MHz to 10 MHz. In some instances, the focal spot, point, and/or region may include (e.g., comprise) a group of neurons.

At 1204, neural activity may be monitored responsive to the radiated ultrasound, e.g., via one or more sensing electrodes positioned on the probe. The neural activity may be associated with patient behavior, at least in some instances. The one or more sensing electrodes may be configured to sense local field potentials. Thus, in at least some instances, the one or more sensing electrodes may be one or more local field potential sensors.

At 1206, a location and/or size of a focal spot or region of the radiated ultrasound may be steered, e.g., based on the monitoring.

At 1208, a treatment zone, e.g., a group and/or volume of neurons, within the acoustic medium may be identified, e.g., based on the steering. In other words, as the location and/or size of the focal spot or region of the radiated ultrasound is steered through the acoustic medium, patient behavior/response may be monitored (e.g., via sensed neural activity and/or observation) to determine and/or identify the group and/or volume of neurons for treatment.

In some instances, an electrical field in the acoustic medium may be generated, e.g., via one or more electrical transducers. In such instances, the neural activity sensed responsive to the radiated ultrasound may also include neural activity sensed responsive to the generated electrical field, e.g., via local field potential sensors. In some instances, the one or more electrical transducers may be configured for electrical deep brain stimulation. Further, in some instances, the ultrasonic transducers and the one or more electrical transducers may share at least one electrode.

In some instances, the array of ultrasonic transducers and local field potential sensors may be included (e.g., comprised) on a probe, e.g., on an ultrasonic probe. In some instances, a probe diameter (and/or diameter of the probe) may be approximately one twenty fourth of a probe length. Further, in some instances, the local field potential sensors may also be included (e.g., comprised) on the probe. In some instances, the ultrasonic probe may be flexible. In other instances, the ultrasonic probe may be approximately and/or substantially rigid, e.g., at least in comparison to the acoustic medium. In some instances, the ultrasonic probe may be made, at least partially, of (e.g., comprised, at least partially, of) acoustically active material. In such instances, the ultrasonic probe may further include a multi-lumen tube and the array of ultrasonic transducers may be comprised on a substrate that resides in an interior of the multi-lumen tube. Additionally, a volume between the substrate and an interior wall of the multi-lumen tube may be filled with an acoustic substance configured to provide acoustic coupling between the substrate and the multi-lumen tube. In some instances, an ultrasonic transducer of the array of ultrasonic transducers may include an electrode pair. In other words, each ultrasonic transducer of the array of ultrasonic transducers may be and/or include an electrode pair. In some instances, the ultrasonic probe may include one or more electrical transducers.

In some instances, a location of the focal spot or region of the radiated ultrasound may be on or in immediate proximity of a surface of the probe thereby enabling substantially direct stimulation and excitation of a small group of neurons. In addition, the location may coincide with a location of at least one sensing electrode of the one or more sensing electrodes.

Embodiments of the present disclosure may be realized in any of various forms. For example, some embodiments may be realized as a computer-implemented method, a computer-readable memory medium (e.g., a non-transitory computer-readable memory medium), and/or a computer system. Other embodiments may be realized using one or more custom-designed hardware devices such as ASICs. Still other embodiments may be realized using one or more programmable hardware elements such as FPGAs.

In some embodiments, a non-transitory computer-readable memory medium may be configured so that it stores program instructions and/or data, where the program instructions, if and/or when executed by a computer system, cause the computer system to perform a method, e.g., any of the method embodiments described herein, or, any combination of the method embodiments described herein, or, any subset of any of the method embodiments described herein, or, any combination of such subsets.

In some embodiments, a computer program, if and/or when executed by a computer system, may cause the computer system to perform a method, e.g., any of the method embodiments described herein, or, any combination of the method embodiments described herein, or, any subset of any of the method embodiments described herein, or, any combination of such subsets.

In some embodiments, a device may be configured to include a processor (or a set of processors) and a memory medium, where the memory medium stores program instructions or a computer program, where the processor is configured to read and execute the program instructions or computer program from the memory medium, where the program instructions are, or computer program is, executable to implement a method, e.g., any of the various method embodiments described herein (or, any combination of the method embodiments described herein, or, any subset of any of the method embodiments described herein, or, any combination of such subsets). The device may be realized in any of various forms.

Although the embodiments above have been described in considerable detail, numerous variations and modifications will become apparent to those skilled in the art once the above disclosure is fully appreciated. It is intended that the following claims be interpreted to embrace all such variations and modifications.

We claim:

1. A method for intra-acoustic medium ultrasonic neuromodulation, comprising:
radiating, via an array of ultrasonic transducers positioned within an acoustic medium, ultrasound through the acoustic medium, wherein a location and/or size of a focal spot or region of the radiated ultrasound is based, at least in part, on input signals to each ultrasonic transducer in the array of ultrasonic transducers; and
sensing, via one or more sensing electrodes positioned within the acoustic medium, neural activity responsive to the radiated ultrasound.

2. The method of claim 1,
wherein the array of ultrasonic transducers and the one or more sensing electrodes are comprised on a probe, and wherein a probe diameter is approximately one twenty fourth of a probe length.

3. The method claim 1,
wherein a location of the focal spot or region is dependent on a length, L, of the array of ultrasonic transducers, and wherein the location of the focal spot or region is between 0.1 L and 2 L from the array of ultrasonic transducers.

4. The method of claim 1,
wherein the one or more sensing electrodes are configured to sense local field potentials.

5. The method of claim 1,
wherein the focal spot or region comprises a group of neurons.

6. The method of claim 1,
wherein ultrasonic transducers in the array of ultrasonic transducers are spaced longitudinally and radially about a cylindrical probe.

7. The method of claim 1, further comprising:
generating, via one or more electrical transducers, an electrical field in the acoustic medium, wherein the one or more electrical transducers are configured for electrical deep brain stimulation; and
wherein sensing, via the one or more sensing electrodes, neural activity responsive to the radiated ultrasound further comprises, sensing, via the one or more sensing electrodes, neural activity responsive to the generated electrical field.

8. The method of claim 7,
wherein the array of ultrasonic transducers, the one or more sensing electrodes, and the one or more electrical transducers are comprised on a probe.

9. The method of claim 7,
wherein the ultrasonic transducers and the one or more electrical transducers share at least one electrode.

10. The method of claim 1,
wherein each ultrasonic transducer in the array of ultrasonic transducers is individually addressable; and
wherein each ultrasonic transducer in the array of ultrasonic transducers is independently controlled to produce the radiated ultrasound.

11. An ultrasonic probe, comprising:
an array of ultrasonic transducers; and
one or more sensing electrodes; and
wherein the ultrasonic probe is configured for intra-acoustic medium ultrasonic modulation via insertion into an acoustic medium, and wherein the ultrasonic probe is configured to:
radiate, via the array of ultrasonic transducers positioned within the acoustic medium, ultrasound through the acoustic medium, wherein a location and/or size of a focal spot or region of the radiated ultrasound is based, at least in part, on input signals to each ultrasonic transducer in the array of ultrasonic transducers; and
sense, via the one or more sensing electrodes, neural activity responsive to the radiated ultrasound.

12. The ultrasonic probe of claim 11,
wherein the ultrasonic probe is flexible.

13. The ultrasonic probe of claim 11,
wherein the ultrasonic probe is at least partially comprised of acoustically active material.

14. The ultrasonic probe of claim 11,
wherein the ultrasonic probe further comprises a multi-lumen tube;
wherein the array of ultrasonic transducers is comprised on a substrate; and
wherein the substrate resides in an interior of the multi-lumen tube.

15. The ultrasonic probe of claim 14,
wherein a volume between the substrate and an interior wall of the multi-lumen tube is filled with an acoustic substance configured to provide acoustic coupling between the substrate and the multi-lumen tube.

16. The ultrasonic probe of claim 11,
wherein a location of the focal spot or region of the radiated ultrasound is on or in immediate proximity of a surface of the probe thereby enabling substantially direct stimulation and excitation of a small group of neurons, and wherein the location coincides with a location of at least one sensing electrode of the one or more sensing electrodes.

17. The ultrasonic probe of claim 11, further comprising:
one or more local field potential sensors; and
one or more electrical transducers, wherein the one or more electrical transducers are configured for electrical deep brain stimulation; and
wherein the ultrasonic probe is further configured to:
generate, via one or more electrical transducers, an electrical field in the acoustic medium; and
sense, via the one or more local field potential sensors, neural activity responsive to the generated electrical field.

18. A non-transitory computer-readable memory medium storing program instructions executable by at least one processor to:
radiate, via an array of ultrasonic transducers arranged on a probe inserted into an acoustic medium, ultrasound through the acoustic medium;
monitor, via one or more sensing electrodes positioned on the probe, neural activity responsive to the radiated ultrasound, wherein the neural activity is associated with patient behavior;
steer, based on the monitoring, a location and/or size of a focal spot or region of the radiated ultrasound; and
identify, based on the steering, a treatment zone within the acoustic medium.

19. The non-transitory computer-readable memory medium of claim 18,
wherein the location and/or size of the focal spot or region of the radiated ultrasound is based, at least in part, on input signals to each ultrasonic transducer in the array of ultrasonic transducers.

20. The non-transitory computer-readable memory medium of claim 18,
wherein the location of the focal spot or region of the radiated ultrasound is dependent on a length, L, of the array of ultrasonic transducers, and wherein the location of the focal spot or region is between 0.1 L and 2 L from the array of ultrasonic transducers.
